(19) United States
(12) Patent Application Publication
(10) Pub. No.: US 2021/0116618 A1
(43) Pub. Date: Apr. 22, 2021

(54) WORK SUPPORT SYSTEM AND PROTECTIVE EYEGLASSES

(71) Applicant: NEC Corporation, Tokyo (JP)

(72) Inventors: Satoshi KYOSUNA, Tokyo (JP); Hirofumi TSUDA, Tokyo (JP); Fujio OKUMURA, Tokyo (JP)

(73) Assignee: NEC Corporation, Tokyo (JP)

(21) Appl. No.: 16/498,089

(22) PCT Filed: Mar. 20, 2018
(86) PCT No.: PCT/JP2018/011036

§ 371 Date: Sep. 26, 2019

(30) Foreign Application Priority Data
Mar. 31, 2017 (JP) 2017-070225

(51) Int. Cl. G02B 5/30; G02F 1/13

(52) U.S. Cl. CPC G02B 5/3025; G02F 1/13

(57) ABSTRACT

A work support system, including: projector which projects circularly polarized light that rotates in a helix centered about a given optical axis in one direction and travels in a first direction perpendicular to a plane of rotation; projection target disposed against the first direction and diffusely reflects the circularly polarized light projected by the projector; and eyeglasses including a pair of lenses which block, when disposed against the first direction in which the circularly polarized light projected from the projector travels, the circularly polarized light traveling in the first direction, and allow, when disposed against a second direction perpendicular to a rotating plane on which circularly polarized light that is part of the light diffusely reflected by the projection target rotating in a direction opposite to the first direction of a helix centered around a given optical axis travels, the circularly polarized light traveling in the second direction to pass.

### TECHNICAL FIELD

The present invention relates to a work support system and protective eyeglasses.

### BACKGROUND ART

In recent years, in order to eliminate human errors in a task at a factory, a store, a delivery base, or the like, or in order to reduce working hours and the associated operating cost, work support systems utilizing projection light have been employed. Specifically, such a work support system captures a work object present at a work site with a camera, recognize captured image, and irradiate the work object on which an operation is to be performed with projection light for giving an instruction to a worker. Using the work support system, by irradiating the work object with the projection light in accordance with the operation from a location distant from the work site, the worker can certainly have visual contact with the work object. Employing the work support system utilizing projection light achieves an effect, in addition to improving work efficiency, that even an unskilled worker can easily perform a task of a level similar to a skilled worker by following the instruction by using the projection light. Therefore, such the work support system has drawn attention as a system for facilitating a variety of tasks existing in the modern world.

As the projection light utilized in the work support system, there has been used laser light with which light can be emitted from a light source (projection light output port). The work support system needs to start with capturing an image of a work object and recognizing the captured image of the work object. Then, the work support system needs to irradiate, in the image of the recognized work object, an object on which an operation is to be performed with laser light. As a device used in the work support system, there has been proposed a device including a combination of a camera and a projection and display device (hereinafter, referred to as a projector).

### CITATION LIST

### Patent Literature

[PTL 1] International Publication No. WO 2012/039267
[PTL 2] Japanese Unexamined Patent Application Publication No. 2013-020079

### Non Patent Literature

[NPL 1] “General-purpose laser safety goggles/protective eyeglasses”, [Online], Newport Corp., [search made on Mar. 15, 2017], the Internet <URL: http://www.laser-concierge.com/product_img/Safety/Newport/Goggle/New port_Goggle.pdf>

### SUMMARY OF INVENTION

### Technical Problem

At the work site, in order for the worker to certainly have visual contact with the work object irradiated with laser light, the laser light with which the work object is irradiated needs to have a certain degree of illuminance. However, in the Ordinance on Industrial Safety and Health, which stipulates criteria for occupational safety and health, it is stipulated to secure a predetermined illuminance (in lux) according to a category of the task at a working surface in a location where the worker regularly works.

Therefore, in order to enable the worker to certainly have visual contact with the work object irradiated with laser light, it is required that the laser light with which the work object is irradiated has light intensity that gives illuminance greater than the illuminance stipulated in the Ordinance on Industrial Safety and Health. On the other hand, even when laser light with a low output level is directly looked, there is a risk of blindness; therefore, care should be taken when handling laser light. For example, the safety guidelines for laser products and users of the laser products defined by International Electrotechnical Commission (IEC) stipulates classification according to output of laser light, wavelengths of laser light, and the like. In addition, it is required to organize an administrative structure for managing occupational safety and health according to class.

In order to reliably protect eyes of the worker who have visual contact with the object irradiated with laser light from the laser light, there is commonly known, for example, protective goggles for laser light described in NPL 1. Typical protective goggles for laser light are equipped with some type of color filter for attenuating intensity of light in the laser wavelength region. Therefore, when the worker wears the protective goggles, the worker sees an image formed on a projection plane by projecting laser light with attenuated intensity of the laser light with which the work object is irradiated. Accordingly, the worker wearing the protective goggles for laser cannot have visual contact with the laser light with which the work object is irradiated. As a result, the worker cannot perform a task in accordance with a projected instruction utilizing the laser light. Therefore, it is not practical to cause a worker who engages in a task using the work support system utilizing laser light to wear the protective goggles for laser.

In short, while the protective goggles for laser can protect eyes of the worker, the protective goggles have a problem in which it is not appropriate for the work support system utilizing laser light.

However, as described above, there is a risk, when laser light is directly looked, the laser light harms eyes. Therefore, it has been desired to develop protective eyeglasses that enable a worker to certainly have visual contact with a work object irradiated with laser light and protect eyes of the worker at the same time.

In view of the problem described above, an object of the present invention is to provide a work support system and protective eyeglasses that solve the problem and enable a worker to certainly have visual contact with a work object irradiated with laser light and protect eyes of the worker at the same time.

### Solution to Problem

A work support system of the present invention includes: a projection means which projects circularly polarized light that rotates in a helix centered about a given optical axis in one direction and travels in a first direction perpendicular to a plane of rotation; a projection target means which is disposed against the first direction and diffusely reflects the circularly polarized light projected from the projection means; and an eyeglass means including a pair of lenses which block, when disposed against the first direction in which the circularly polarized light projected from the projection means travels, the circularly polarized light traveling in the first direction, and allow, when disposed against a second direction perpendicular to a rotating plane on which circularly polarized light that is part of the light diffusely reflected by the projection target means rotating in a direction opposite to the first direction of a helix centered around a given optical axis travels, the circularly polarized light traveling in the second direction to pass.

Protective eyeglasses of the present invention includes a pair of lenses which block, when disposed against a first direction in which circularly polarized light projected from a projection means which projects circularly polarized light travels, the circularly polarized light rotating in a helix centered about a given optical axis in one direction, and that is perpendicular to a plane of rotation, the circularly polarized light traveling in the first direction, and allow, when disposed against a second direction perpendicular to a rotating plane on which circularly polarized light that is part of the light diffusely reflected by a projection target means that is disposed against the first direction and diffusely reflects the circularly polarized light projected from the projection means rotating in a direction opposite to the first direction of a helix centered around a given optical axis travels, the circularly polarized light traveling in the second direction to pass.

### Advantageous Effects of Invention

The present invention can provide a work support system and protective eyeglasses that enable a worker to certainly have visual contact with laser light with which a work object is irradiated and protect eyes of the worker at the same time.

### BRIEF DESCRIPTION OF DRAWINGS

FIG. 1 is a block diagram illustrating an example of a phase-modulation type projector constituting a work support system according to a first example embodiment of the present invention;

FIG. 2 is a block diagram illustrating an example of an irradiation unit of the phase-modulation type projector constituting the work support system according to the first example embodiment;

FIG. 3 is a diagram for describing an example of a relationship between projection light and polarized light when looking light, which is projected from the phase-modulation type projector constituting the work support system according to the first example embodiment and reflected by a projection target surface, with protective eyeglasses;

FIG. 4 is a diagram for describing an example of a relationship between projection light and polarized light when looking light projected from the phase-modulation type projector constituting the work support system according to the first example embodiment with protective eyeglasses;

FIG. 5 is a perspective view illustrating an example of an exterior appearance of the protective eyeglasses constituting the work support system according to the first example embodiment;

FIG. 6 is a sectional view of the protective eyeglasses taken along A-A′ in FIG. 5 when a base plate (lens) of the protective eyeglasses constituting the work support system according to the first example embodiment is plastic;

FIG. 7 is a sectional view of the protective eyeglasses taken along A-A′ in FIG. 5 when the base plate (lens) of the protective eyeglasses constituting the work support system according to the first example embodiment is glass; and

FIG. 8 is a schematic block diagram illustrating an example of a work support system according to a second example embodiment of the present invention.

### EXAMPLE EMBODIMENT

Before describing a first example embodiment of the present invention, an outline of a work support system according to the example embodiment of the present invention will be described.

The work support system according to the present example embodiment includes a phase-modulation type projector and protective eyeglasses. First, the phase-modulation type projector will be described.

Techniques relating to projectors have been remarkably developed. A projector is a type of display device. The projector is a projection and display device for displaying an image or a video by projecting the image or video on a projection target surface such as a large screen.

Modulation methods of projectors include an intensity-modulation type and a phase-modulation type. The phase-modulation type has a characteristic that a power efficiency thereof is better than that of the intensity-modulation type. The phase-modulation type may be designed, even if the projector is distant from a screen, such that an image to be displayed on the projection target surface does not look dim. The phase-modulation type generates a Fourier transformed image of an image to be projected on high-resolution liquid crystal elements, controls phases for pixels according to changes in refractive index and obtains the image to be projected.

Laser light emitted by a phase-modulation type projector having such a characteristic is projected only at an area in which the image to be projected on a projection target surface is formed. In other words, the laser light emitted by the phase-modulation type projector is not projected at an area in which the image is not formed. In other words, the laser light emitted by the phase-modulation type projector has a characteristic that it is not a so-called diffused light. Therefore, energy of the laser light emitted by the phase-modulation type projector is delivered only to the area in which the image is formed (an illuminated area), and the energy is not delivered to the area in which the image is not formed (a dim area). Consequently, even though intensity of light that laser light provide is proportional to a size of the projector, the phase-modulation type projector can emit brighter laser light compared to a projector of a different type with lower energy. This is one reason that a phase-modulation type projector is employed in the work support system according to the present example embodiment. Other reasons will be described later.

### First Example Embodiment

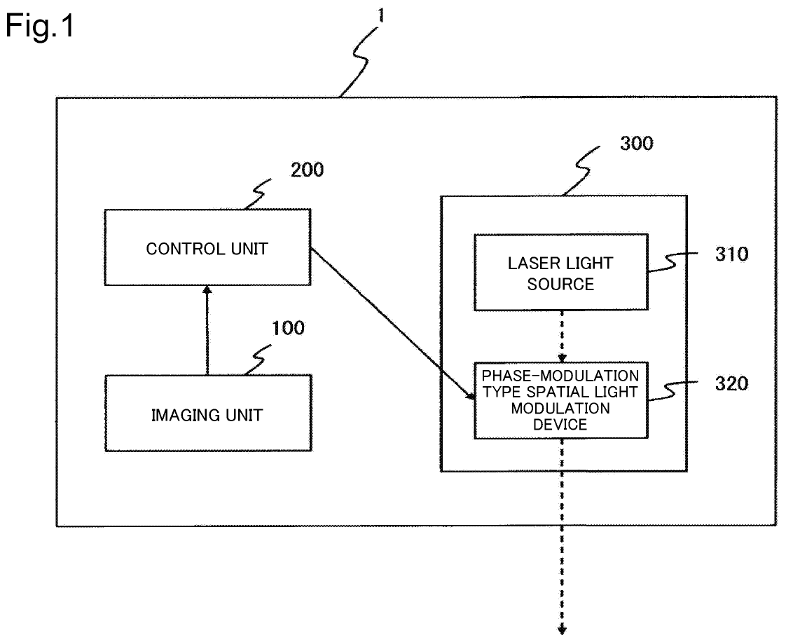

Next, the phase-modulation type projector constituting the work support system according to the first example embodiment of the present invention will be described by using FIG. 1. FIG. 1 is a block diagram illustrating an example of the phase-modulation type projector constituting the work support system according to the present example embodiment. In FIG. 1, a dotted line indicates a transmission of laser light while a solid line indicates a flow of information. Note that a unidirectional arrow in the figure described below simply indicates a flow of a certain signal (data), and it does not exclude bidirectionality.

Referring to FIG. 1, a phase-modulation type projector 1 constituting the work support system according to the present example embodiment includes an imaging unit 100, a control unit 200, and an irradiation unit 300. Each of them will be individually described below.

The imaging unit 100 captures an image of an object existing outside the phase-modulation type projector 1. Information on the object imaged by the imaging unit 100, motion of the object, or the like (hereinafter, these are collectively referred to as “object or the like”) is stored in an unillustrated storage unit of the phase-modulation type projector 1. The imaging unit 100 is achieved using, for example, an imaging element such as a complementary metal-oxide semiconductor (CMOS) device, a three-dimensional depth detecting element, or the like.

The control unit 200 identifies or recognizes, using image processing such as pattern recognition, the object or the like captured by the imaging unit 100 (hereinafter, identification is not discriminated from recognition and referred to as “recognition”). The control unit 200 controls a phase-modulation type spatial light modulation device 320 on the basis of the recognition result. In other words, the control unit 200 determines an image to be radiated by the irradiation unit 300 based on the recognition result. The control unit 200 controls the phase-modulation type spatial light modulation device 320 such that an image formed by light output by the phase-modulation type spatial light modulation device 320 corresponds to the image determined by the control unit 200.

The irradiation unit 300 includes a laser light source 310 and the phase-modulation type spatial light modulation device 320. The laser light source 310 includes a component for emitting laser light. The laser light source 310 and the phase-modulation type spatial light modulation device 320 are disposed in such a way that the laser light emitted by the laser light source 310 enters the phase-modulation type spatial light modulation device 320. The phase-modulation type spatial light modulation device 320 has a function of modulating, in response to incident laser light, a phase of the laser light and outputting the laser light.

The irradiation unit 300 may further include an image-forming optical system to be described later, an unillustrated irradiation optical system, or the like. The irradiation unit 300 projects an image formed by the light output by the phase-modulation type spatial light modulation device 320.

The control unit 200 and the phase-modulation type spatial light modulation device 320 of the present example embodiment will be further described. The phase-modulation type spatial light modulation device 320 is achieved using a diffractive-optical element of a phase-modulation type.

The phase-modulation type spatial light modulation device 320 includes a plurality of light receiving regions. The light receiving region is a cell constituting the phase-modulation type spatial light modulation device 320. The light receiving regions are arranged, for example, in a one-dimensional or two-dimensional array. The control unit 200 controls, based on control information, each of a plurality of the light receiving regions constituting the phase-modulation type spatial light modulation device 320 in such a way that a parameter that determines a difference between a phase of light incident on the light receiving region and a phase of light to be output from the light receiving region changes. Specifically, the control unit 200 controls each of a plurality of the light receiving regions, for example, in such a way that an optical property such as a refractive index or an optical path length changes. A distribution of phases of light incident on the phase-modulation type spatial light modulation device 320 changes according to change in the optical property of each light receiving region. Accordingly, the phase-modulation type spatial light modulation device 320 outputs light representing the control information.

The phase-modulation type spatial light modulation device 320 includes, for example, ferroelectric liquid crystals, homogeneous liquid crystals, vertically oriented liquid crystals, or the like. The phase-modulation type spatial light modulation device 320 is achieved, for example, using a liquid crystal on silicon (LCOS) technique. In this case, the control unit 200 controls, for each of a plurality of the light receiving regions constituting the phase-modulation type spatial light modulation device 320, a voltage to be applied to the light receiving region. The refractive index of the light receiving region changes according to the applied voltage. Accordingly, the control unit 200 can generate, by controlling the refractive index of each of the light receiving regions constituting the phase-modulation type spatial light modulation device 320, a difference in the refractive index among the light receiving regions. In the phase-modulation type spatial light modulation device 320, in accordance with the control by the control unit 200, incident laser light is appropriately diffracted by each of the light receiving regions.

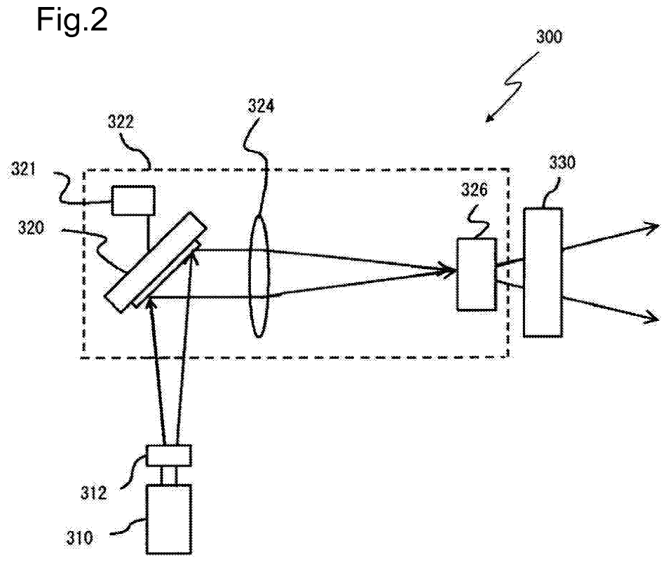

FIG. 2 is a block diagram illustrating an example of an irradiation unit of the phase-modulation type projector constituting the work support system according to the first example embodiment.

The irradiation unit 300 of the phase-modulation type projector constituting the work support system according to the present example embodiment includes, in addition to the laser light source 310, a shaping optical system 312, a projection control unit 322, and a projection unit 330.

The shaping optical system 312 is disposed at a light output port of the laser light source 310. The shaping optical system 312 shapes the light emitted by the laser light source 310 into a shape suitable for processes to be performed subsequently, i.e., phase modulation, Fourier transform, image formation, and projection. A polarization state of the light emitted by the laser light source 310 is stored until the light is projected from the projection unit 330. The light output from the shaping optical system 312 enters a light receiving surface of the phase-modulation type spatial light modulation device 320 in the projection control unit 322.

The projection control unit 322 includes the phase-modulation type spatial light modulation device 320, a modulation control unit 321, a Fourier transform lens 324, and an image-forming optical system 326.

The phase-modulation type spatial light modulation device 320 phase-modulates light incident on the projection control unit 322.

The modulation control unit 321 changes the refractive indexes of a plurality of the light receiving regions included in the phase-modulation type spatial light modulation device 320 according to information, for each pixel, on the image to be projected.

The light that has been phase-modulated using the phase-modulation type spatial light modulation device 320 is diffracted upon passing through the Fourier transform lens 324. The Fourier transform lens 324 performs Fourier transform on the light that has been phase-modulated using the phase-modulation type spatial light modulation device 320. The light diffracted after passing through the Fourier transform lens 324 concentrates toward the image-forming optical system 326. The image-forming optical system 326 focuses the light that has passed through the Fourier transform lens 324. For example, the image-forming optical system 326 focuses light that has passed through an unillustrated polarization preserving element. The image-forming optical system 326 inputs the focused light into the projection unit 330. The projection unit 330 projects the light input by the image-forming optical system 326.

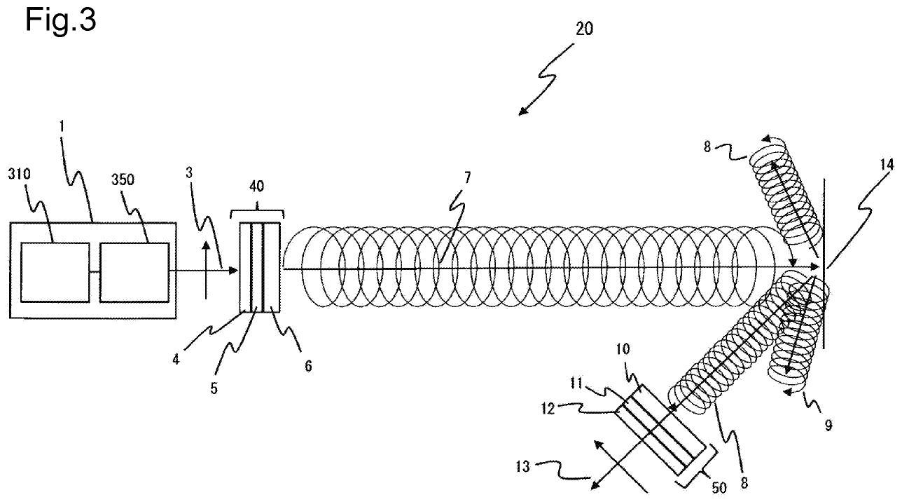

Next, an example of a relationship between projection light and polarized light when looking light, which is projected from the phase-modulation type projector constituting the work support system according to the first example embodiment and reflected by the projection target surface, with protective eyeglasses will be described with reference to FIG. 3.

Referring to FIG. 3, a work support system 20 according to the present example embodiment includes the phase-modulation type projector 1, a projector-side polarizing member 40, a projection target surface 14, and a protective eyeglasses-side polarizing member 50.

The phase-modulation type projector 1 is the one describe above in FIG. 1 and FIG. 2. The phase-modulation type projector 1 includes the laser light source 310 and an optical system 350 including the irradiation unit 300.

The projector-side polarizing member 40 includes a linearly polarizing plate 4, a clockwise circularly polarizing plate 5, and a protective glass 6.

The protective eyeglasses-side polarizing member 50 includes a protective glass 10, a counterclockwise circularly polarizing plate 11, and a linearly polarizing plate 12.

Next, fundamentals of the work support system according to the present example embodiment will be described. As a projection system of the work support system 20, a phase-modulation type such as the phase-modulation type projector 1 is assumed. The projection system has a characteristic that its output is linearly polarized. Laser light output from the laser light source 310 of the phase-modulation type projector 1 is input into the optical system 350. From the optical system 350, linearly polarized output light 3 is output. The output light is circularly polarized using a polarization system of the projector-side polarizing member 40. The linearly polarized output light 3 enters the linearly polarizing plate 4. The linearly polarized output light 3 that has passed through the linearly polarizing plate 4 enters the clockwise circularly polarizing plate 5. The light that has passed through the clockwise circularly polarizing plate 5 and has been circularly polarized in a clockwise direction enters the non-polarizing protective glass 6 that does not change polarization of light. The light that has passed through the protective glass 6 and has been circularly polarized in a clockwise direction serves as a clockwise circularly polarized projection light 7 and travels toward the projection target surface 14.

Note that, depending on a degree of polarization of the output light 3 from the phase-modulation type projector 1, when the output light 3 from the phase-modulation type projector 1 has a sufficient degree of linear polarization, for example, when the degree of linear polarization is 100%, the linearly polarizing plate 4 of the projector-side polarizing member 40 may be omitted.

If the output light 3 from the phase-modulation type projector 1 is non-polarized and the output light 3 is caused to pass through the projector-side polarizing member 40, light intensity of the output light after passing through the polarizing member 40 decreases by half. However, as described in FIG. 1 and FIG. 2, the phase-modulation type projector 1 can emit brighter laser light compared to a projector of a different type with lower energy. Therefore, when using the phase-modulation type projector 1, loss in light intensity is small and sufficient brightness can be ensured.

The clockwise circularly polarized projection light 7 that has traveled toward the projection target surface 14 is diffusely reflected by the projection target surface 14. The clockwise circularly polarized projection light 7 is reflected by the projection target surface 14 and turns into clockwise circularly polarized reflected light 9 and counterclockwise circularly reflected light 8. In this case, it is assumed that the clockwise circularly polarized reflected light 9 and the counterclockwise circularly reflected light 8 travel toward eyes of a worker wearing protective eyeglasses 2 to be described later. Note that, as described later, in FIG. 3, a pair of lenses of the protective eyeglasses 2 worn by the worker includes the counterclockwise circularly polarizing plate 11. Therefore, the clockwise circularly polarized reflected light 9 does not reach the eyes of the worker.

The protective eyeglasses-side polarizing member 50 represents a structure of the lenses of the protective eyeglasses 2 to be described later. First, the counterclockwise circularly polarized reflected light 8 enters the protective glass 10 of the protective eyeglasses 2. The protective glass 10 may be the lens itself of the eyeglasses equipped with a glass lens. The counterclockwise circularly polarized reflected light 8 that has passed through the protective glass 10 enters the counterclockwise circularly polarizing plate 11. The counterclockwise circularly polarized reflected light 8 that has passed through the counterclockwise circularly polarizing plate 11 enters the linearly polarizing plate 12. The counterclockwise circularly polarized reflected light 8 that has passed through the linearly polarizing plate 12 is converted into linearly polarized light. The linearly polarized reflected light 13 enters the eyes of the worker. In short, when a polarization direction of linearly polarized light and a polarization direction of the linearly polarizing plate 12 are the same, the linearly polarized light passes through the linearly polarizing plate 12; when the directions are perpendicular to each other, the linearly polarized light is blocked.

The worker wears the protective eyeglasses 2 including a pair of lenses that constitute a polarization system (the protective eyeglasses-side polarizing member 50) composed of a circularly polarizing plate that polarizes light in a direction opposite to the polarization direction (in FIG. 3, clockwise circular polarization) of the projection light obtained by circularly polarizing the light output from the phase-modulation type projector 1 and a linearly polarizing plate. In short, in the protective eyeglasses 2 worn by the worker, adjustment of angles of the linearly polarizing plate and the circularly polarizing plate is performed in such a way that only light having the same direction of rotation as the counterclockwise circularly polarized reflected light 8 that has been diffusely reflected by the projection target surface 14 passes through the protective eyeglasses 2. For example, in a case of a task to be performed at a factory, a store, a delivery base, or the like, in terms of ensuring safety of a worker, it is considered that there are many workplaces where wearing eyeglasses is mandatory in order to protect eyes of the worker. Therefore, it is considered that wearing the protective eyeglasses 2 constituting the work support system 20 according to the present example embodiment does not lead to uncomfortable feeling.

Although an eyesight tends to be dim when the protective eyeglasses 2 are worn, a pupil of an eye opens more; therefore, one will not visually feel dark. As described above, the present example embodiment achieves effects of ensuring visibility of laser light and protection of eyes of the worker.

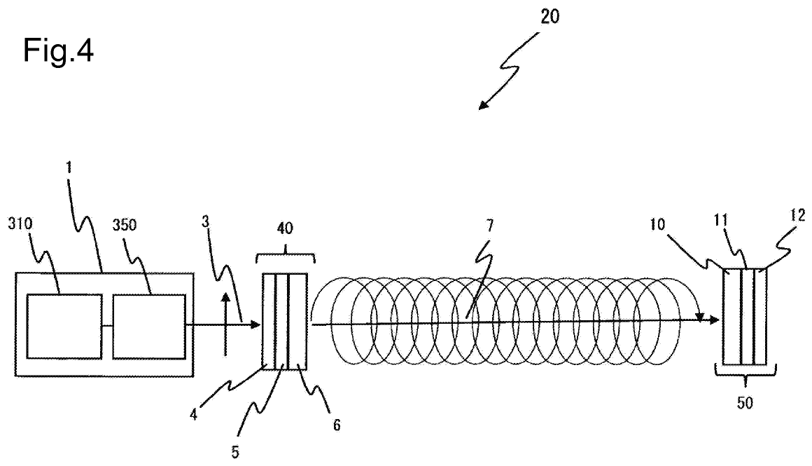

FIG. 4 is a diagram for describing an example of a relationship between projection light and polarized light when looking light projected from the phase-modulation type projector constituting the work support system according to the first example embodiment with protective eyeglasses.

In the work support system 20 illustrated in FIG. 4, there is illustrated a state in which the protective eyeglasses 2 are worn and laser light output from a projection light output port of the phase-modulation type projector 1 is directly looked. In this case, the clockwise circularly polarized projection light 7 that is output from the phase-modulation type projector 1 and that has passed through the projector-side polarizing member 40 enters the protective eyeglasses-side polarizing member 50. In the protective eyeglasses 2, counterclockwise circularly polarizing plates 11 are provided for both eyes. Accordingly, the clockwise circularly polarized projection light 7 is blocked by the protective eyeglasses 2. Therefore, intensity of laser light entering the eyes of the worker is reduced to a level that causes little harm. When an experiment was conducted with emitting light corresponding to class 2 defined in the safety guidelines for laser devices and the users thereof by the International Electrotechnical Commission (IEC), light intensity merely as high as that of a lighted light emitting diode (LED) could be felt. Note that class 2 is defined specifically for visible light, and the light used in this case corresponds to a laser for which eye protection is afforded by an aversion response such as an eye blink and safety corresponding to class 1 (a level that is safe for eyes) is ensured.

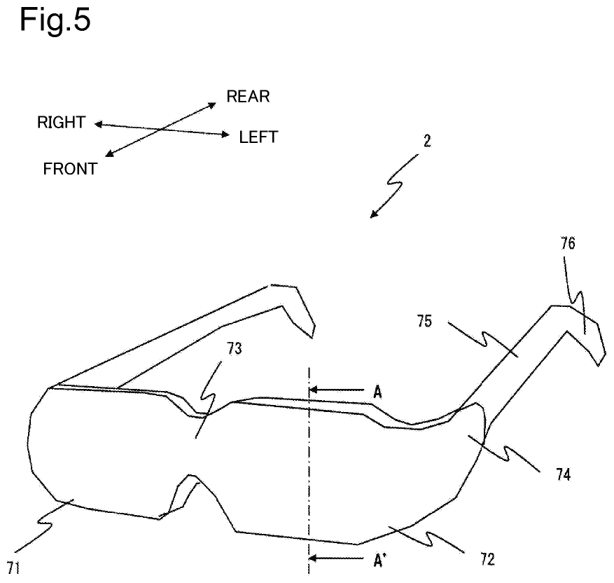

Next, an exterior appearance of the protective eyeglasses constituting the work support system according to the first example embodiment will be described. FIG. 5 is a perspective view illustrating an example of the exterior appearance of the protective eyeglasses constituting the work support system according to the first example embodiment. Arrows in FIG. 5 respectively represent front, rear, leftward, and rightward directions of the protective eyeglasses 2.

In FIG. 5, the protective eyeglasses 2 include a right lens 71, a left lens 72, an end piece 74, a temple 75, and a temple tip.

In addition, the protective eyeglasses 2 constituting the work support system according to the present example embodiment includes a counterclockwise circularly polarizing plate 11 for both of the right lens 71 and the left lens 72. The counterclockwise circularly polarizing plate 11 transmits the counterclockwise circularly polarized reflected light 8 obtained by polarizing the linearly polarized output light 3 output by the phase-modulation type projector 1 with the clockwise circularly polarizing plate 5 and diffusely reflecting the resultant clockwise circularly polarized projection light 7 with the projection target surface 14. The counterclockwise circularly polarizing plate 11 blocks the clockwise circularly polarized projection light 7 obtained by polarizing the linearly polarized output light 3 output by the phase-modulation type projector 1 with the clockwise circularly polarizing plate 5.

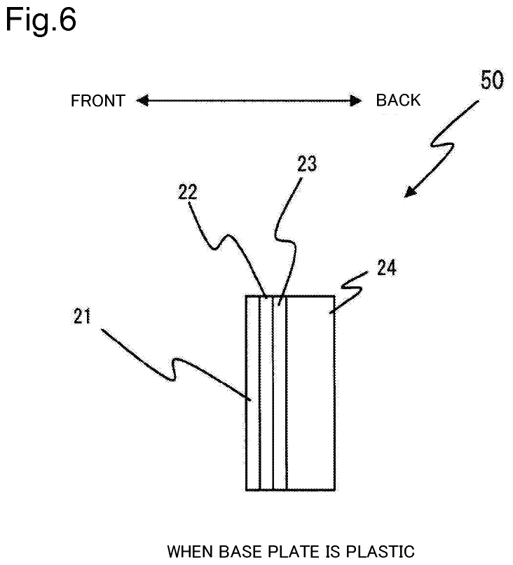

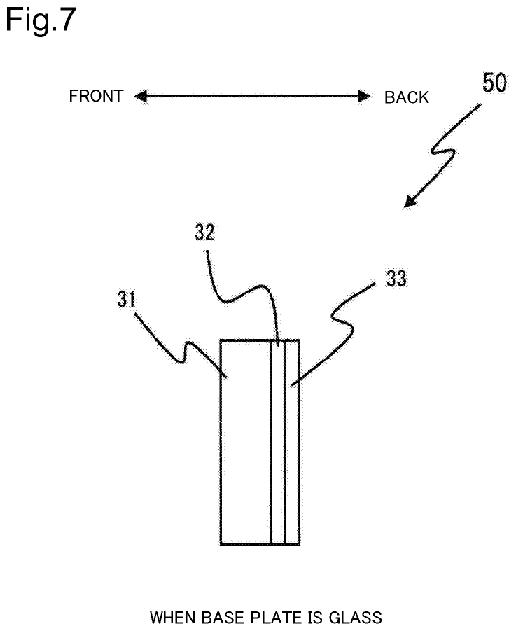

Next, internal structures of the protective eyeglasses in a case in which a base plate (lens) of the protective eyeglasses constituting the work support system according to the first example embodiment is plastic and in a case in which the base plate is glass will be described. FIG. 6 is a sectional view of the protective eyeglasses taken along A-A′ in FIG. 5 when the base plate (lens) of the protective eyeglasses constituting the work support system according to the first example embodiment is plastic. FIG. 7 is a sectional view of the protective eyeglasses taken along A-A′ in FIG. 5 when the base plate (lens) of the protective eyeglasses is glass.

In FIG. 6, the protective eyeglasses-side polarizing member 50 includes, in order from the front of the protective eyeglasses 2, a thin-plate tempered glass 21, a circularly polarizing plate 22, a linearly polarizing plate 23, and a plastic base plate 24.

In the example, it is assumed that the base plate, i.e., the base plate of the lens inherently provided in the eyeglasses is plastic. In other words, when the lenses of the eyeglasses are made of a commonly used plastic material, it is highly likely that the plastic material adversely affects polarized light. Therefore, plates of the polarization system (the circularly polarizing plate 22 and the linearly polarizing plate 23) are preferably provided ahead of the plastic base plate 24.

However, in this structure, there is a problem in which a surface of the circularly polarizing plate 22 may be easily damaged. For example, in a workplace, such as a work site for welding, where iron dust flies, the surface of the circularly polarizing plate 22 may be immediately damaged, and there may be potentially generated a portion through which light may pass. Therefore, on the surface of the circularly polarizing plate 22, the thin-plate tempered glass 21 as thick as several tens of micrometers is provided. A plate as thick as this can be bent like paper. Furthermore, up to a curved surface including the end piece 74 of the protective eyeglasses 2, the surface of the circularly polarizing plate 22 can be protected using the thin-plate tempered glass 21.

In FIG. 7, the protective eyeglasses-side polarizing member 50 includes, in order from the front of the protective eyeglasses 2, a glass base plate 31, a circularly polarizing plate 32, and a linearly polarizing plate 33.

In the example, it is assumed that the base plate, i.e., the base plate of the lens inherently provided in the eyeglasses is glass. In the structure, the glass base plate 31 (lens) serves to protect the surface of the circularly polarizing plate 32. In this manner, when the base plate (lens) of the eyeglasses is glass, plates of the polarization system (the circularly polarizing plate 32 and the linearly polarizing plate 33) can be provided inside (rearward).

As described above, by performing a simple processing on the lens inherently provided in the eyeglasses, the protective eyeglasses 2 constituting the work support system 20 according to the present example embodiment can be achieved. Furthermore, by attaching the plate of the polarization system illustrated in FIG. 6 or FIG. 7 to an unprocessed plastic eyeglass lens or glass eyeglass lens before the lens is attached to the eyeglass frame, a lens dedicated to the protective eyeglasses 2 constituting the work support system 20 according to the present example embodiment can be made.

Next, a difference between the work support system according to the example embodiment of the present invention and a so-called head-mounted display as well as an effect of solving a problem that may not be solved by the head-mounted display and that is specific to the present invention will be described. The head-mounted display is a type of wearable computer, and it is a display device mounted on a head. When constructing the work support system according to the example embodiment of the present invention using the head-mounted display, there is a problem described below.

A worker wearing a head-mounted display first attempts to have visual contact with an image indicating a work object displayed on the head-mounted display. The worker then performs a task while keeping visual contact with the actual work object. In other words, between identifying the work object and actually performing the task, the worker compares the work object displayed on the head-mounted display and the actual object.

As a result, a line of sight of the worker moves. Therefore, there is a problem in which the worker cannot certainly have visual contact with the work object. As described, the head-mounted display cannot solve the problem in which the worker cannot certainly have visual contact with the work object, which is a problem that the present invention intends to solve.

Next, a difference between the work support system according to the example embodiment of the present invention and a so-called 3D system as well as an effect of solving a problem that may not be solved by the 3D system and that is specific to the present invention will be described. RealD (a registered trademark), which is an example of the 3D system, is widespread as a 3D system for movie theaters, and it employs a circular polarization method. In the 3D system at movie theaters, a polarizing plate is disposed ahead of a lens of a motion-picture projector.

The system circularly polarizes frames for right eyes in a clockwise direction. In addition, the system circularly polarizes frames for left eyes in a counterclockwise direction. An audience seeing pictures wears eyeglasses, in order to see frames individually specified for left or right eyes, the eyeglasses in which a clockwise circularly polarizing lens is mounted for the right eye and a counterclockwise circularly polarizing lens is mounted for the left eye. In short, the 3D system has a characteristic that the audience sees pictures achieved by light reflected by a special screen that does not adversely affect polarized light using eyeglasses that individually transmit clockwise and counterclockwise polarized light. In this manner, a three-dimensional stereoscopic effect, which enables the audience to see the pictures as if the pictures extend in backward and forward directions with respect to the screen, is achieved.

The 3D system and the work support system according to the example embodiment of the present invention are different in the following points. That is, the screen at movie theaters used for the 3D system is a special screen that does not adversely affect polarized light projected from a motion-picture projector. Compared to this, the laser light emitted by the phase-modulation type projector in the work support system according to the example embodiment of the present invention is diffusely reflected by the projection target surface. Therefore, clockwise and counterclockwise circularly polarized light are diffusely reflected by the projection target surface. In addition, in the present example embodiment, light that has been adversely affected by the diffusing surface (diffusely reflecting surface) is looked using the eyeglasses with a pair of lenses each including a rotationally polarizing plate having the same rotational polarization direction. Specifically, laser light is looked using the eyeglasses with a pair of lenses each having a rotational polarization direction opposite to the direction of rotational polarization of the projection system.

It is assumed here that, in a state in which the work object is irradiated with laser light according to the present example embodiment, the eyeglasses employed in the 3D system are used. In this case, if counterclockwise circularly polarized light enters the clockwise circularly polarizing lens and clockwise circularly polarized light enters the counterclockwise circularly polarizing lens, there arises a problem in which the laser light cannot be entirely seen. As a result, the problem in which the worker cannot certainly have visual contact with the work object, which is a problem that the present invention intends to solve, cannot be solved.

In addition, when directly looking laser light, if clockwise circularly polarized light enters the clockwise circularly polarizing lens and counterclockwise circularly polarized light enters the counterclockwise circularly polarizing lens, the laser light squarely enters eyes. As a result, protecting eyes of a worker, which is a challenge that the present invention intends to solve, cannot be solved.

As described above, the work support system according to the present invention can solve the problems that can never be solved by techniques for the head-mounted display or techniques for the 3D system, which are commonly used, at once.

The present invention uses a projection device for outputting linearly polarized light and a projection system for generating circularly polarized light by causing the output light to pass through a linearly polarizing plate and a circularly polarizing plate and projecting the resultant light. For protective eyeglasses worn by a worker who potentially sees a projected image near the projection device, a polarization system including a combination of the circularly polarizing plate and the linearly polarizing plate is provided. Furthermore, a rotational direction of circular polarization of the protective eyeglasses is set to a direction opposite to that of the projection system. Since the worker near the projection device wears the protective eyeglasses, light directly emitted by the projection device is significantly attenuated by the polarization system of the protective eyeglasses. As a result, eyes of the worker can be protected. In addition, by wearing the protective eyeglasses, the worker can have visual contact with laser light that is projected from the projection device and reflected. As a result, the worker can certainly have visual contact with the laser light with which the work object is irradiated.

In the example embodiment described above, description has been made taking the case in which the linearly polarized light projected from the phase-modulation type projector 1 is circularly polarized in a clockwise direction as an example. However, the linearly polarized light projected from the phase-modulation type projector 1 may be circularly polarized in a counterclockwise direction. In this case, the pair of lenses of the protective eyeglasses 2 is constructed using a circularly polarizing plate that circularly polarizes light in the clockwise direction.

### Second Example Embodiment

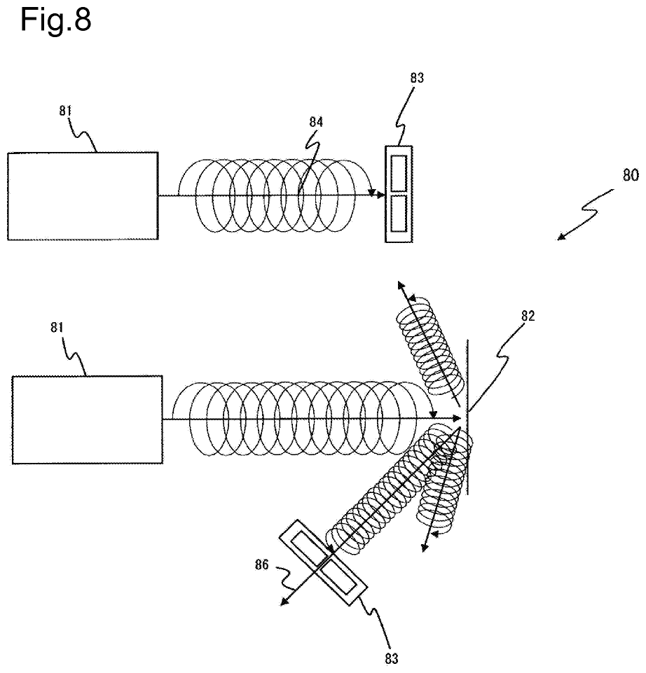

Next, a second example embodiment of the present invention will be described. FIG. 8 is a diagram illustrating an example of a work support system according to the second example embodiment of the present invention.

Referring to FIG. 8, the work support system 80 according to the present example embodiment includes a projection unit 81, a projection target unit 82, and eyeglasses 83.

The projection unit 81 projects circularly polarized light that rotates in a helix centered about a given optical axis in one direction and travels in a first direction 84 perpendicular to a plane of rotation.

The projection target unit 82 is disposed against the first direction 84 and diffusely reflects the circularly polarized light projected from the projection unit 81.

The eyeglasses 83 include a pair of lenses that blocks, when disposed against the first direction 84 in which the circularly polarized light projected from the projection unit 81 travels, circularly polarized light traveling in the first direction 84. The eyeglasses 83 further include a pair of lenses that transmits, when disposed against a second direction 86 in which a portion of light diffusely reflected by the projection target unit 82 travels, the portion rotating in a helix centered about a given optical axis in a direction opposite to the one direction and circularly polarized, and that is perpendicular to a plane of rotation, circularly polarized light traveling in the second direction 86.

Note that an alternative means of the phase-modulation type spatial light modulation device 320 described above may be achieved by using a micro electro mechanical system (MEMS) technique, for example.

In addition, a computer program stored in the unillustrated storage unit of the phase-modulation type projector may be provided in a form of a recording medium, or may be provided via a network such as the Internet. The recording medium is a computer-usable medium or a computer-readable medium, and it includes a medium that allows information to be recorded or read magnetically, optically, electronically, electromagnetically, or by means of infrared light or the like. Such a medium includes, for example, a semiconductor memory device, a storage device using a semiconductor device or a solid object, a magnetic tape, a removable computer diskette, random access memory (RAM), read only memory (ROM), a magnetic disk, an optical disc, a magneto-optical disc, and the like.

The present invention has been described above with reference to the example embodiments; however, the present invention is not limited to the aforementioned example embodiments. Various modifications that could be understood by those skilled in the art may be made to the configurations or details of the present invention within the scope of the present invention.

Some or all of the aforementioned example embodiments may be described as in the following supplementary notes, but are not limited to the following.

### (Supplementary Note 1)

A work support system, including:

a projection means which projects circularly polarized light that rotates in a helix centered about a given optical axis in one direction and travels in a first direction perpendicular to a plane of rotation;

a projection target means which is disposed against the first direction and diffusely reflects the circularly polarized light projected from the projection means; and

an eyeglass means including a pair of lenses which
block, when disposed against the first direction in which the circularly polarized light projected from the projection means travels, circularly polarized light traveling in the first direction, and
allow, when disposed against a second direction perpendicular to a rotating plane on which circularly polarized light that is part of the light diffusely reflected by the projection target means rotating in a direction opposite to the first direction of a helix centered around a given optical axis travels, the circularly polarized light traveling in the second direction to pass.

### (Supplementary Note 2)

The work support system according to supplementary note 1, wherein

the pair of lenses includes, in order from a side against the first or second direction, a circularly polarizing member that converts polarization of light into circular polarization and a linearly polarizing member that converts polarization of light into linear polarization, and

the pair of lenses
block, when a polarization direction of the light that has been converted into linearly polarized light by the linearly polarizing member and a polarization direction of the linearly polarizing member are perpendicular, circularly polarized light traveling in the first or second direction and
transmit, when the polarization direction of the light that has been converted into linearly polarized light by the linearly polarizing member and the polarization direction of the linearly polarizing member are the same, circularly polarized light traveling in the first or second direction.

### (Supplementary Note 3)

The work support system according to supplementary note 1 or 2, wherein

the projection means includes
a phase-modulation type projection means which projects linearly polarized light and
a circularly polarizing conversion means which converts the linearly polarized light projected from the phase-modulation type projection means into circularly polarized light traveling in the first direction.

### (Supplementary Note 4)

The work support system according to any one of supplementary notes 1 to 3, wherein the projection means projects circularly polarized light traveling in the first direction through a non-polarizing member that does not change polarization of light.

### (Supplementary Note 5)

The work support system according to any one of supplementary notes 1 to 4, wherein a side against the first direction and a side against the second direction of the pair of lenses are covered with a non-polarizing member that does not change polarization of light.

### (Supplementary Note 6)

The work support system according to supplementary notes 5, wherein the non-polarizing member is glass.

### (Supplementary Note 7)

Protective eyeglasses including a pair of lenses which
block, when disposed against a first direction in which circularly polarized light projected from a projection means for projecting circularly polarized light travels, the circularly polarized light rotating in a helix centered about a given optical axis in the first direction, and that is perpendicular to a plane of rotation, circularly polarized light traveling in the first direction, and
allow, when disposed against a second direction perpendicular to a rotating plane on which circularly polarized light that is part of the light diffusely reflected by projection target means that is disposed against the first direction and diffusely reflects the circularly polarized light projected from the projection means rotating in a direction opposite to the first direction of a helix centered around a given optical axis travels, the circularly polarized light traveling in the second direction to pass.

### (Supplementary Note 8)

The protective eyeglasses according to supplementary note 7, wherein

the pair of lenses includes, in order from a side against the first or second direction, a circularly polarizing member that converts polarization of light into circular polarization and a linearly polarizing member that converts polarization of light into linear polarization, and

the pair of lenses
block, when a polarization direction of the light that has been converted into linearly polarized light by the linearly polarizing member and a polarization direction of the linearly polarizing member are perpendicular, circularly polarized light traveling in the first or second direction and
transmit, when the polarization direction of the light that has been converted into linearly polarized light by the linearly polarizing member and the polarization direction of the linearly polarizing member are the same, circularly polarized light traveling in the first or second direction.

### (Supplementary Note 9)

The protective eyeglasses according to supplementary note 7 or 8, wherein

the projection means includes
a phase-modulation type projection means which projects linearly polarized light and
a circularly polarizing conversion means which converts the linearly polarized light projected from the phase-modulation type projection means into circularly polarized light traveling in the first direction.

### (Supplementary Note 10)

The protective eyeglasses according to any one of supplementary notes 7 to 9, wherein the projection means projects circularly polarized light traveling in the first direction through a non-polarizing member that does not change polarization of light.

### (Supplementary Note 11)

The protective eyeglasses according to any one of supplementary notes 7 to 10, wherein a side against the first direction and a side against the second direction of the pair of lenses are covered with a non-polarizing member that does not change polarization of light.

### (Supplementary Note 12)

The protective eyeglasses according to supplementary notes 11, wherein the non-polarizing member is glass.

This application is based upon and claims the benefit of priority from Japanese Patent Application No. 2017-070225, filed on Mar. 31, 2017, the disclosure of which is incorporated herein in its entirety by reference.

### REFERENCE SIGNS LIST

1 Phase-modulation type projector
2 Protective eyeglasses
3 Output light
4, 12 Linearly polarizing plate
5 Clockwise circularly polarizing plate
6, 10 Protective glass
7 Clockwise circularly polarized projection light
8 Counterclockwise circularly polarized reflected light
9 Clockwise circularly polarized reflected light
11 Counterclockwise circularly polarizing plate
13 Reflected light
20, 80 Work support system
21 Thin-plate tempered glass
22, 32 Circularly polarizing plate
23, 33 Linearly polarizing plate
24 Plastic base plate
31 Glass base plate
40 Projector-side polarizing member
50 Protective eyeglasses-side polarizing member
71 Right lens
72 Left lens
73 Bridge
74 End piece
75 Temple
76 Temple tip
81 Projection unit
82 Projection target unit
83 Eyeglasses
84 First direction
86 Second direction
100 Imaging unit
200 Control unit
300 Irradiation unit
310 Laser light source
312 Shaping optical system
320 Phase-modulation type spatial light modulation device
322 Projection control unit
324 Fourier transform lens
326 Image-forming optical system
330 Projection unit

1. A work support system, comprising:
a projection means a projector configured to project circularly polarized light that rotates in a helix centered around a first direction and travels in the first direction;
a projection target configured to be disposed against the first direction and diffusely reflects the circularly polarized light projected from the the projector; and
eyeglasses including a pair of lenses which is configured to
block, when the pair of lenses are disposed against the first direction in which the circularly polarized light projected from the projector travels, the circularly polarized light traveling in the first direction, and
allow circularly polarized light traveling in a second direction to pass through the pair of lenses, when the pair of lenses are disposed against the second direction perpendicular to a rotating plane of the circularly polarized light being part of the light diffusely reflected by the projection target and rotating around the second direction in a direction opposite to the rotation direction of the projected circularly polarized light.

2. The work support system according to claim 1, wherein
the pair of lenses includes, in order from a side against the first or second direction, a circularly polarizing member that converts polarization of light into circular polarization and a linearly polarizing member that converts polarization of light into linear polarization, and
the pair of lenses is configured to
block, when a polarization direction of the light that has been converted into linearly polarized light by the linearly polarizing member and a polarization direction of the linearly polarizing member are perpendicular, the circularly polarized light traveling in the first or second direction and
transmit, when the polarization direction of the light that has been converted into linearly polarized light by the linearly polarizing member and the polarization direction of the linearly polarizing member are the same, the circularly polarized light traveling in the first or second direction.

3. The work support system according to claim 1, wherein
the projector includes
phase-modulation type projection unit which is configure to project linearly polarized light and
circularly polarizing conversion unit which is configure to convert the linearly polarized light projected from the phase-modulation type projection unit into circularly polarized light traveling in the first direction.

4. The work support system according to claim 1, wherein the projector is configured to project circularly polarized light traveling in the first direction through a non-polarizing member that does not change polarization of light.

5. The work support system according to claim 1, wherein a side against the first direction and a side against the second direction of the pair of lenses are covered with a non-polarizing member that does not change polarization of light.

6. The work support system according to claim 5, wherein the non-polarizing member is glass.

7. Protective eyeglasses comprising a pair of lenses which are configured to
block, when the pair of lenses are disposed against a first direction in which circularly polarized light projected from a projector for projecting circularly polarized light travels, the circularly polarized light rotating in a helix centered about a given optical axis in one direction, and that is perpendicular to a plane of rotation, the circularly polarized light traveling in the first direction, and
allow circularly polarized light traveling in a second direction to pass through the pair of lenses, when the pair of lenses are disposed against the second direction perpendicular to a rotating plane of the circularly polarized light being part of the light diffusely reflected by a projection target that is disposed against the first direction and diffusely reflects the circularly polarized light projected from the projector and rotating around the second direction in a direction opposite to the rotation direction of the projected circularly polarized light.

8. The protective eyeglasses according to claim 7, wherein
the pair of lenses includes, in order from a side against the first or second direction, a circularly polarizing member that converts polarization of light into circular polarization and a linearly polarizing member that converts polarization of light into linear polarization, and
the pair of lenses is configured to
block, when a polarization direction of the light that has been converted into linearly polarized light by the linearly polarizing member and a polarization direction of the linearly polarizing member are perpendicular, the circularly polarized light traveling in the first or second direction and
transmit, when the polarization direction of the light that has been converted into linearly polarized light by the linearly polarizing member and the polarization direction of the linearly polarizing member are the same, the circularly polarized light traveling in the first or second direction.

9. The protective eyeglasses according to claim 7, wherein
the projector includes
a phase-modulation type projection unit which is configured to project linearly polarized light and
a circularly polarizing conversion unit which is configured to convert the linearly polarized light projected from the phase-modulation type projection unit into circularly polarized light traveling in the first direction.

10. The protective eyeglasses according to claim 7, wherein the projector is configured to project circularly polarized light traveling in the first direction through a non-polarizing member that does not change polarization of light.

11. The protective eyeglasses according to claim 7, wherein a side against the first direction and a side against the second direction of the pair of lenses are covered with a non-polarizing member that does not change polarization of light.

12. The protective eyeglasses according to claim 11, wherein the non-polarizing member is glass.
